(19) United States
(12) Patent Application Publication
(10) Pub. No.: US 2024/0417535 A1
(43) Pub. Date: Dec. 19, 2024

(54) HYDROPHOBIC AND SELF-CLEANING RESIN SLURRY, AND HYDROPHOBIC AND SELF-CLEANING RESIN DENTAL MATERIAL AND USE THEREOF

(71) Applicant: ANHUI MEDICAL UNIVERSITY, Hefei, Anhui (CN)

(72) Inventors: Shunli ZHENG, Hefei (CN); Ling WENG, Hefei (CN); Qin RAO, Hefei (CN); Xiangyang LI, Hefei (CN); Quanli LI, Hefei (CN); Ying CAO, Hefei (CN); Jialong CHEN, Hefei (CN); Wenjuan ZHANG, Hefei (CN); Siyuan CHEN, Hefei (CN); Le ZHANG, Hefei (CN); Xiaowei WANG, Hefei (CN)

(73) Assignee: ANHUI MEDICAL UNIVERSITY, Hefei, Anhui (CN)

(21) Appl. No.: 18/017,599

(22) PCT Filed: Nov. 3, 2022
(86) PCT No.: PCT/CN2022/129534

§ 371 Date: Jan. 23, 2023

(30) Foreign Application Priority Data
Sep. 27, 2021 (CN) 202111134716.7

(51) Int. Cl. C08K 3/36; A61K 6/17; A61K 6/62; A61K 6/871; A61K 6/887; C08F 2/50; C08F 220/28; C08K 5/05; C08K 5/17

(52) U.S. Cl. CPC C08K 3/36; A61K 6/17; A61K 6/62; A61K 6/871; A61K 6/887; C08F 2/50; C08F 220/285; C08K 5/05; C08K 5/175; C08K 2201/005; C08K 2201/011

(57) ABSTRACT

Provided area hydrophobic and self-cleaning resin slurry, and a hydrophobic and self-cleaning resin dental material and use thereof, which relate to the technical field of dental materials. In the disclosure, the hydrophobic and self-cleaning resin slurry includes the following raw materials: a dimethacrylate monomer, triethylene glycol dimethacrylate, perfluoroalkyl acrylate, a diluent, nano-silica, γ-methacryloxypropyltrimethoxysilane, camphorquinone, and a photo-curing accelerator.

### CROSS REFERENCE TO RELATED APPLICATION

The present application claims priority to the Chinese Patent Application No. CN202111134716.7, filed with the China National Intellectual Property Administration (CNIPA) on Sep. 27, 2021, and entitled “HYDROPHOBIC AND SELF-CLEANING RESIN SLURRY, AND HYDROPHOBIC AND SELF-CLEANING RESIN DENTAL MATERIAL AND USE THEREOF”, the application of which is incorporated herein by reference in its entirety.

### TECHNICAL FIELD

The present disclosure relates to the technical field of dental materials, in particular to a hydrophobic and self-cleaning resin slurry, and a hydrophobic and self-cleaning resin dental material and use thereof.

### BACKGROUND

Caries prevention is a major need of public health in China. Dental caries is also considered to be the most important public health problem and one of the serious burdens of disease treatment resources in the world. Maximizing the effect of caries prevention and treatment has become an urgent problem to be solved in clinical dentistry. The root cause of dental caries is because dental plaque biofilms from a caries-causing bacteria as the dominant microflora colonize on the surface of teeth, decompose carbohydrates in food, produce acids, and continue to repeat the above activities, resulting in demineralization of tooth hard tissues.

Common oral diseases such as dental caries, periodontitis, and local irritation factors of dental calculus are all related to dental plaque biofilms attached to the tooth surface. At present, dental plaque biofilms are most commonly removed by mechanical removal or antibacterial agents clinically. The mechanical removal includes brushing and flossing. However, such physical removal methods lack persistence and can't meet the standard of plaque control. Commonly used antibacterial agents include antibiotics, antibacterial and bactericidal chemical agents (such as chlorhexidine, triclosan, silver agents, and antimicrobial peptides), fluorides, extracts of traditional Chinese medicines and natural plants, and small molecules of enzymes that inhibit metabolism; such antibacterial agents prevent biofilm maturation by means of bacteriostasis, sterilization, inhibition of bacterial adhesion, and disruption of the extracellular matrix of plaque. However, the antibacterial agents as described above do not have self-cleaning and anti-biofouling efficacies.

### SUMMARY

In view of the above, an object of the present disclosure is to provide a hydrophobic and self-cleaning resin slurry, and a hydrophobic and self-cleaning resin dental material and use thereof. In the present disclosure, the hydrophobic and self-cleaning resin slurry and the hydrophobic and self-cleaning resin dental material have self-cleaning and anti-biofouling performances, and can inhibit the formation of bacterial biofilms.

To achieve the above object of the present disclosure, the present disclosure provides the following technical solutions.

The present disclosure provides a hydrophobic and self-cleaning resin slurry, including the following raw materials in parts by weight: 3 parts to 10 parts of a dimethacrylate monomer, 5 parts to 30 parts of triethylene glycol dimethacrylate (TEGDMA), 5 parts to 15 parts of perfluoroalkyl acrylate (FMA), 0 part to 15 parts of a diluent, 1 part to 15 parts of nano-silica, 1 part to 30 parts of γ-methacryloxypropyltrimethoxysilane (KH-570), 0.05 parts to 0.5 parts of camphorquinone (CQ), and 0.1 parts to 1 part of a photo-curing accelerator.

In some embodiments, the dimethacrylate monomer includes one or more selected from the group consisting of bisphenol A-glycerolate dimethacrylate (Bis-GMA) and urethane dimethacrylate (UDMA).

In some embodiments, the photo-curing accelerator includes one or more selected from the group consisting of ethyl 4-dimethylaminobenzoate (EDMAB) and dimethylaminoethyl methacrylate (DMAEMA).

In some embodiments, the nano-silica has a particle size of 15 nm to 20 nm.

The present disclosure further provides a method for preparing the hydrophobic and self-cleaning resin slurry, including the following steps:
mixing the dimethacrylate monomer, the TEGDMA, the FMA, the diluent, the nano-silica, the KH-570, the CQ and the photo-curing accelerator to obtain the hydrophobic and self-cleaning resin slurry.

In some embodiments, the mixing is conducted for 1.5 h to 6 h.

In some embodiments, the mixing specifically includes:
conducting a first mixing on the dimethacrylate monomer, the TEGDMA, the FMA, and the diluent to obtain a first mixture;
conducting a second mixing on the first mixture, the nano-silica, and the KH-570 to obtain a second mixture; and
conducting a third mixing on the second mixture, the CQ, and the photo-curing accelerator.

In some embodiments, the first mixing is conducted for 0.5 h to 2 h; the second mixing is conducted for 0.5 h to 2 h; and the third mixing is conducted for 0.5 h to 2 h.

The present disclosure further provides a hydrophobic and self-cleaning resin dental material, obtained by conducting photo-curing on the hydrophobic and self-cleaning resin slurry as described above or a hydrophobic and self-cleaning resin slurry prepared by the method as described above.

In some embodiments, the photo-curing is conducted at an optical wavelength of 420 nm to 480 nm.

In some embodiments, the photo-curing is conducted for 20 s to 80 s.

The present disclosure further provides use of a hydrophobic and self-cleaning resin slurry as described above, a hydrophobic and self-cleaning resin slurry prepared by the method as described above or the hydrophobic and self-cleaning resin dental material as described above as a dental material.

The present disclosure provides a hydrophobic and self-cleaning resin slurry, including the following raw materials in parts by weight: 3 parts to 10 parts of a dimethacrylate monomer, 5 parts to 30 parts of TEGDMA, 5 parts to 15 parts of FMA, 0 part to 15 parts of a diluent, 1 part to 15 parts of nano-silica, 1 part to 30 parts of KH-570, 0.05 parts to 0.5 parts of CQ, and 0.1 parts to 1 part of a photo-curing accelerator. In the present disclosure, the dimethacrylate monomer is used as a resin matrix, the TEGDMA is used as a diluting monomer, and the FMA with hydrophobic functional groups (long fluorocarbon chains) and resin reactive functional groups (carbon-carbon double bonds) is added as a hydrophobic modifier to reduce the surface energy of the material. The nano-silica is added as an inorganic filler, which not only increases the mechanical properties of the resin, but also improves the microscopic roughness of the resin, thus constructing a lotus leaf-like micro-nano hierarchical structure with excellent hydrophobic properties. A hydrophobic surface of such micro-nano structure can retain air, forming an air layer on the material surface, reducing a contact area with proteins and bacteria (site adhesion), thereby inhibiting the adhesion of oral cavity-related bacteria (such as Streptococcus mutans) and the formation of bacterial biofilm. Accordingly, the material has excellent self-cleaning and anti-biofouling performances (i.e. performances of inhibiting the adhesion of bacteria and protein on a surface). Moreover, one end of the organosilane KH-570 molecule is —Si(OCH₃)₃, which becomes —Si(OH)₃ after hydrolysis; the —Si(OH)₃ can be condensed with —OH on the surface of SiO₂ to form a —Si—O— bond to link with each other, thereby improving a binding force between the inorganic filler nano —SiO₂ and the resin as well as dispersion of the nano —SiO₂ in the resin.

The present disclosure further provides a hydrophobic and self-cleaning resin dental material, obtained by conducting photo-curing on the hydrophobic and self-cleaning resin slurry as described above. During the use, the photosensitizer CQ generates active free radicals through photoactivation in the presence of the photo-curing accelerator, and then initiates the polymerization of carbon-carbon double bonds in the dimethacrylate monomer, the TEGDMA, the FMA, and the KH-570 to form the hydrophobic and self-cleaning resinous dental material. The hydrophobic and self-cleaning resin dental material has a lotus leaf-like micro-nano structure, endowing the hydrophobic and self-cleaning resin dental material with excellent hydrophobic, self-cleaning, and anti-biofouling properties. A hydrophobic surface of the micro-nano structure can retain air, forming an air layer on a surface of the material, reducing a contact area with proteins and bacteria (site adhesion), thereby inhibiting the adhesion of oral cavity-related bacteria (such as Streptococcus mutans) and the formation of bacterial biofilm. In addition, the hydrophobic and self-cleaning resin slurry uses the dimethacrylate monomer as a resin matrix and has desirable biocompatibility.

### BRIEF DESCRIPTION OF THE DRAWINGS

FIG. 1A to FIG. 1F show scanning electron microscopy (SEM) images of resin dental materials according to Example 4 and Comparative Example 1, wherein FIG. 1A to FIG. 1C₃ correspond to Comparative Example 1, and FIG. 1D to FIG. 1F correspond to Example 4;

FIG. 2A to FIG. 2J show a dynamic behavior of water droplets in contact with surfaces of the resin dental materials according to Example 4 and Comparative Example 1, wherein FIG. 2A to FIG. 2E correspond to Comparative Example 1, and FIG. 2F to FIG. 2J correspond to Examples 4;

FIG. 3A to FIG. 3L show fluorescent micrographs of the cell compatibility of extract solutions of the resin dental materials according to Example 4 and Comparative Examples 1 to 2 and a blank control group, wherein FIG. 3A to FIG. 3C correspond to the blank control group, FIG. 3D to FIG. 3F correspond to Comparative Example 2, FIG. 3G to FIG. 3I correspond to Comparative Example 1, and FIG. 3J to FIG. 3L correspond to Example 4; and

FIG. 4A to FIG. 4D show fluorescence staining images of bacterial adhesion on the surface of the resin dental materials according to Example 4 and Comparative Examples 1 to 2 and the blank control group by a laser scanning confocal microscope (LSCM), wherein FIG. 4A corresponds to the blank control group, FIG. 4B corresponds to the Comparative Example 2, FIG. 4C corresponds to the Comparative Example 1, and FIG. 4D corresponds to the Example 4.

### DETAILED DESCRIPTION OF THE EMBODIMENTS

The present disclosure is further described below with reference to the accompanying drawings and embodiments.

The present disclosure provides a hydrophobic and self-cleaning resin slurry, including the following raw materials in parts by weight: 3 parts to 10 parts of a dimethacrylate monomer, 5 parts to 30 parts of TEGDMA, 5 parts to 15 parts of FMA, 0 parts to 15 parts of a diluent, 1 part to 15 parts of nano-silica, 1 part to 30 parts of KH-570, 0.05 parts to 0.5 parts of CQ, and 0.1 parts to 1 part of a photo-curing accelerator.

In the present disclosure, unless otherwise specified, all raw material components are commercially available products well known to persons skilled in the art.

In the present disclosure, the raw material for preparing the hydrophobic and self-cleaning resin slurry includes 3 parts to 10 parts, preferably 4 parts to 9 parts, more preferably 5 parts to 6 parts of the dimethacrylate monomer in parts by weight. In some embodiments the dimethacrylate monomer includes Bis-GMA and/or UDMA. In some embodiments the dimethacrylate monomer includes the Bis-GMA or the UDMA.

In the present disclosure, the Bis-GMA has the following structural formula:

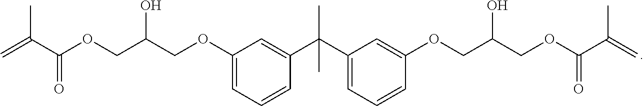 [SMILES: C=C(C)C(=O)OCC(O)COc1cccc(C(C)(C)c2cccc(OCC(O)COC(=O)C(=C)C)c2)c1]

In the present disclosure, the UDMA has the following structural formula:

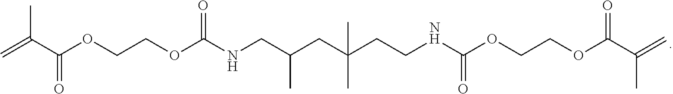 [SMILES: C=C(C)C(=O)OCCOC(=O)NCCC(C)(C)CC(C)CNC(=O)OCCOC(=O)C(=C)C]

In the present disclosure, in parts by weight of the dimethacrylate monomer, the raw material for preparing the hydrophobic and self-cleaning resin slurry includes 5 parts to 30 parts, preferably 8 parts to 25 parts, more preferably 10 parts to 22 parts, most preferably 20 parts of the TEGDMA. In the present disclosure, the TEGDMA has the following structural formula:

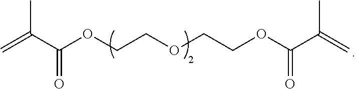

In the present disclosure, in parts by weight of the dimethacrylate monomer, the raw material for preparing the hydrophobic and self-cleaning resin slurry includes 5 parts to 15 parts, preferably 7 parts to 13 parts, more preferably 8 parts to 12 parts, most preferably 10 parts to 11 parts of the FMA. In the present disclosure, the FMA has the following structural formula:

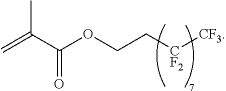

In the present disclosure, in parts by weight of the dimethacrylate monomer, the raw material for preparing the hydrophobic and self-cleaning resin slurry includes 0 part to 15 parts, more preferably 1 part to 12 parts, most preferably 5 parts to 10 parts of diluent. In some embodiments the diluent is an alcohol solvent. In some embodiments the diluent is ethanol.

In the present disclosure, in parts by weight of the dimethacrylate monomer, the raw material for preparing the hydrophobic and self-cleaning resin slurry includes 1 part to 15 parts, preferably 2 parts to 10 parts, more preferably 3 parts to 8 parts, most preferably 4 parts to 5 parts of nano-silica (SiO₂). In some embodiments the nano-silica has a particle size of 15 nm to 20 nm; the nano-silica is hydrophilic and has hydroxyl groups on a surface.

In the present disclosure, in parts by weight of the dimethacrylate monomer, the raw material for preparing the hydrophobic and self-cleaning resin slurry includes 1 part to 30 parts, preferably 5 parts to 20 parts, more preferably 5 parts to 15 parts, most preferably 5 parts to 10 parts of the KH-570. In the present disclosure, the KH-570 has the following structural formula:

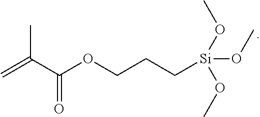 [SMILES: C=C(C)C(=O)OCCC[Si](OC)(OC)OC]

In the present disclosure, in parts by weight of the dimethacrylate monomer, the raw material for preparing the hydrophobic and self-cleaning resin slurry includes 0.05 parts to 0.5 parts, preferably 0.08 parts to 0.4 parts, more preferably 0.09 parts to 0.3 parts, and most preferably 0.1 parts to 0.2 parts of camphorquinone (CQ); in some embodiment, the CQ is DL-CQ (CAS number: 10373-78-1).

In the present disclosure, in parts by weight of the dimethacrylate monomer, the raw material for preparing the hydrophobic and self-cleaning resin slurry includes 0.1 parts to 1 part, preferably 0.2 parts to 0.8 parts, more preferably 0.3 parts to 0.4 parts of a photo-curing accelerator. In some embodiments the photo-curing accelerator includes ethyl 4-dimethylaminobenzoate (EDMAB) and/or dimethylaminoethyl methacrylate (DMAEMA). In some embodiment the photo-curing accelerator includes the EDMAB or the DMAEMA.

The present disclosure further provides a method for preparing the hydrophobic and self-cleaning resin slurry as described above, including the following steps:
mixing the dimethacrylate monomer, the TEGDMA, the FMA, the diluent, the nano-silica, the KH-570, the CQ, and the photo-curing accelerator to obtain the hydrophobic and self-cleaning resin slurry.

In some embodiments of the present disclosure, the mixing is conducted in the dark; in some embodiments, the mixing is conducted in a brown bottle. In some embodiments of the present disclosure, the mixing is conducted for preferably 1.5 h to 6 h, more preferably 3 h to 4.5 h at room temperature by stirring; and there is no special limitation on a stirring speed, as long as the raw materials could be mixed evenly.

In the present disclosure, in some embodiments the mixing includes: conducting a first mixing on the dimethacrylate monomer, the TEGDMA, the FMA, and the diluent to obtain a first mixture; conducting a second mixing on the first mixture, the nano-silica, and the KH-570 to obtain a second mixture; and conducting a third mixing on the second mixture, the CQ, and the photo-curing accelerator to obtain the hydrophobic and self-cleaning resin slurry. The first mixing is conducted for preferably 0.5 h to 2 h, more preferably 1 h to 1.5 h. In some embodiments the KH-570 is added dropwise; there is no special limitation on a speed of the adding dropwise, and the agent can be added drop by drop; the second mixing starts timing when the KH-570 is completely added dropwise; and the second mixing is conducted for preferably 0.5 h to 2 h, more preferably 1 h to 1.5 h. The third mixing is conducted for preferably 0.5 h to 2 h, more preferably 1 h to 1.5 h.

The present disclosure further provides a hydrophobic and self-cleaning resin dental material, obtained by conducting photo-curing on the hydrophobic and self-cleaning resin slurry. In some embodiments, the photo-curing is conducted under anaerobic conditions, at an optical wavelength of preferably 420 nm to 480 nm, more preferably 430 nm to 470 nm, and most preferably 450 nm to 460 nm, for preferably 20 s to 80 s, more preferably 30 s to 70 s, and most preferably 40 s to 60 s, with preferably a dental photo-curing machine. During the photo-curing, the CQ generates active free radicals through photoactivation in the presence of the photo-curing accelerator, and then initiates the polymerization of carbon-carbon double bonds in the dimethacrylate monomer, the TEGDMA, the FMA, and the KH-570 to form the hydrophobic and self-cleaning resin dental material with high biosafety and a lotus leaf-like micro-nano hierarchical structure.

The hydrophobic and self-cleaning resin dental material has a lotus leaf-like micro-nano structure, endowing the material with excellent hydrophobic, self-cleaning, and anti-biofouling properties. A hydrophobic surface of the micro-nano structure can retain air, forming an air layer on the material surface, reducing a contact area with proteins and bacteria (site adhesion), thereby inhibiting the adhesion of oral cavity-related bacteria (such as Streptococcus mutans) and the formation of bacterial biofilm. Therefore, it can be used as a prevention and treatment material for dental plaque-related diseases from the perspective of source etiology. Compared with the long-term application of currently-used bactericidal/bacteriostatic agents that can cause side effects such as flora imbalance and drug resistance, the hydrophobic and self-cleaning resin dental material does not target the microorganisms themselves, and does not interfere with the oral micro-ecosystem or cause related drug-resistant problems. In addition, the hydrophobic and self-cleaning resin dental material uses the dimethacrylate monomer as a resin matrix and has desirable biocompatibility.

The present disclosure further provides use of the hydrophobic and self-cleaning resin slurry or the hydrophobic and self-cleaning resin dental material as a dental material. In the present disclosure, in some embodiments the hydrophobic and self-cleaning resin slurry or the hydrophobic and self-cleaning resin dental material is used as a self-cleaning coating, a restoration, a denture, or an implant surface layer. In some embodiments the self-cleaning coating includes a tooth surface self-cleaning coating and a denture surface self-cleaning coating; and the restoration includes a dentition defect restoration or a dental caries restoration. The hydrophobic and self-cleaning resin dental material has a lotus leaf-like micro-nano structure, showing excellent hydrophobic properties, desirable biocompatibility, and “anti-biofouling” self-cleaning performances that inhibit bacterial adhesion, which can be used as a dental material. Moreover, the hydrophobic and self-cleaning resin slurry can be coated in a liquid state to achieve in-situ curing in the oral cavity, which has the advantages of clinical application efficiency and indications. As a result, the slurry can be applied to a special oral environment, such as caries repair and prevention materials for senile root caries and xerostomia patients.

The technical solutions of the present disclosure will be described below clearly and completely in conjunction with the embodiments of the present disclosure. Apparently, the described embodiments are only a part of, not all of, the embodiments of the present disclosure. All other embodiments obtained by those of ordinary skill in the art based on the embodiments of the present disclosure without creative efforts shall fall within the protection scope of the present disclosure.

### Example 1

(1) 0.5 g of Bis-GMA, 1.5 g of TEGDMA, 1.0 g of FMA, and 0.5 g of absolute ethanol were added into a brown glass bottle, and stirred magnetically for 0.5 h at room temperature to obtain a first mixture.

(2) 0.1 g of nano-silica (SiO₂, with a particle size of 15 nm to 20 nm) was added into the brown glass bottle, added with 0.1 g of KH-570 dropwise under magnetic stirring, and then stirred magnetically at room temperature for 0.5 h to obtain a second mixture.

(3) 0.0105 g of CQ and 0.02405 g of EDMAB were added into the brown glass bottle, and magnetically stirred for 1 h at room temperature to obtain a hydrophobic and self-cleaning resin slurry.

(4) The hydrophobic and self-cleaning resin slurry obtained in step (3) was photo-cured by a dental photo-curing machine under anaerobic conditions for 20 s to obtain a hydrophobic and self-cleaning resin dental material.

### Examples 2 to 5

The hydrophobic and self-cleaning resin dental material was prepared according to the method of Example 1, and preparation conditions of Examples 2 to 5 were shown in Table 1.

TABLE 1
Preparation conditions of Examples 1 to 5

| | Bis-GMA | TEGDMA | FMA | SiO₂ | | KH-570 | | CQ | EDMAB | Photo-curing time |
|---|---|---|---|---|---|---|---|---|---|---|
| Example 1 | 0.5 g | 1.5 g | 1.0 g | 0.1 | g | 0.1 | g | 0.0105 g | 0.02405 g | 20 sec |
| Example 2 | 0.5 g | 1.0 g | 0.5 g | 0.25 | g | 0.25 | g | 0.0105 g | 0.02045 g | 40 sec |
| Example 3 | 0.8 g | 1.0 g | 0.5 g | 0.3 | g | 0.3 | g | 0.0105 g | 0.02405 g | 60 sec |
| Example 4 | 0.5 g | 2.0 g | 1.0 g | 0.5 | g | 0.5 | g | 0.0105 g | 0.02045 g | 80 sec |
| Example 5 | 0.5 g | 2.0 g | 1.0 g | 1.0 | g | 1.0 | g | 0.0105 g | 0.02405 g | 80 sec |

### Comparative Example 1

(1) 0.5 g of Bis-GMA, 2.0 g of TEGDMA, and 0.5 g of absolute ethanol were added into a brown glass bottle, and stirred magnetically for 0.5 h at room temperature to obtain a first mixture.

(2) 0.0075 g of CQ and 0.0175 g of EDMAB were added into the brown glass bottle, and magnetically stirred for 1 h at room temperature to obtain a hydrophilic resin slurry.

(3) The hydrophilic resin slurry obtained in step (2) was photo-cured by a dental photo-curing machine under anaerobic conditions for 40 s to obtain a hydrophilic resin dental material.

### Comparative Example 2

A commercial resin dental material (3M Filtek™ P60) was photo-cured by a dental photo-curing machine for 20 s under anaerobic conditions to obtain a cured commercial resin dental material.

### Test Example

Taking Example 4 as an example, the surface morphology and properties were studied for the hydrophobic and self-cleaning resin dental material provided by the present disclosure.

### (1) Microscopic Morphology and Surface Wettability

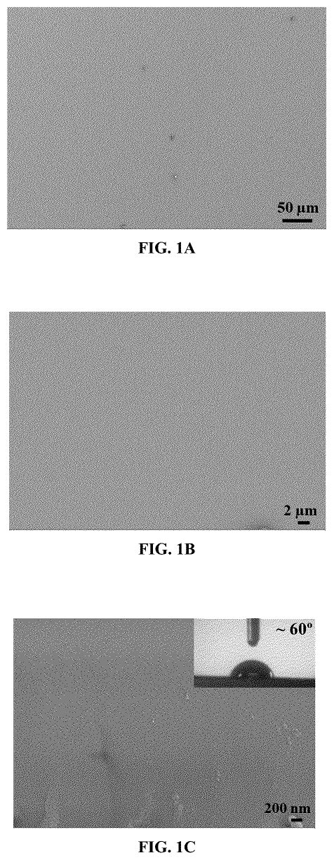

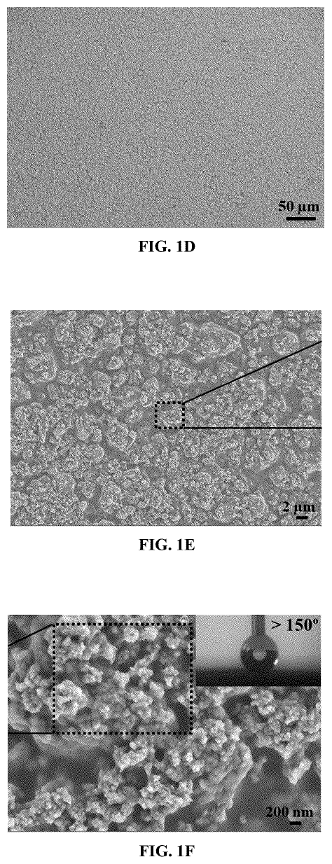

FIG. 1A to FIG. 1F show SEM images of resin dental materials according to Example 4 and Comparative Example 1, wherein FIG. 1A to FIG. 1C correspond to Comparative Example 1, and FIG. 1D to FIG. 1F correspond to Example 4. It can be seen from FIG. 1D to FIG. 1F that the hydrophobic and self-cleaning resin dental material according to Example 4 is composed of many micron-scale protrusion structures; many nano —SiO₂ particles are distributed on a surface of the micron-scale protrusion structure, constituting a micro-nano hydrophobic surface with a lotus leaf-like structure (at a contact angle of greater than) 150°. Compared with the relatively smooth hydrophilic resin dental materials (FIG. 1A to FIG. 1C, at a contact angle of about) 60°, a hydrophobic surface of the micro-nano structure could retain air, forming an air layer on the material surface, and reducing a contact area with bacteria (site adhesion), thereby inhibiting the formation of bacterial biofilms.

### (2) Dynamic Behavior of Material Surface Contact

A test method: 5 μL of water droplet were placed on a surface of the resin dental materials according to Example 4 and Comparative Example 1, the resin dental materials were lifted up by hands and a pressure was applied to the water droplet, and then the resin dental materials were moved downward.

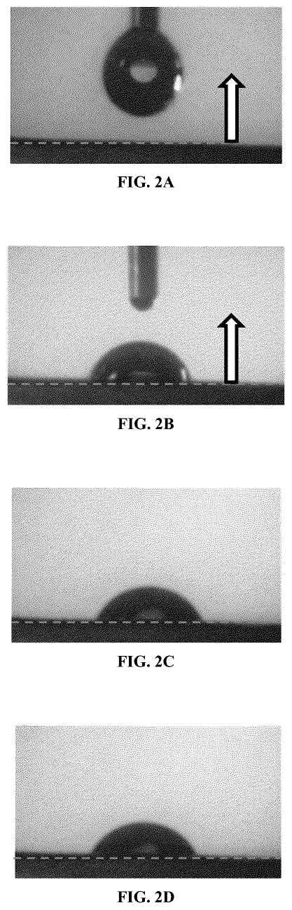

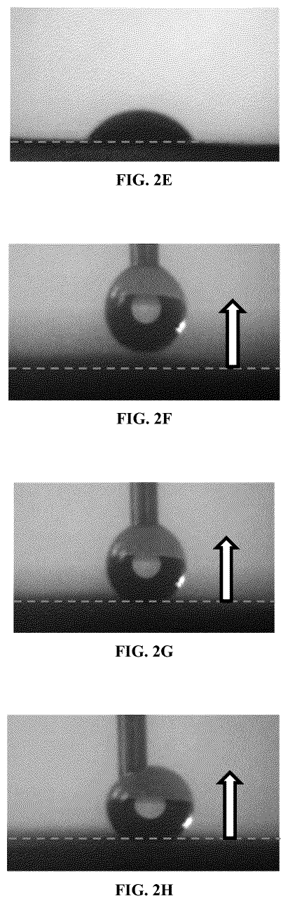

FIG. 2A to FIG. 2J show a dynamic behavior of water droplets in contact with the surface of the resin dental materials according to Example 4 and Comparative Example 1, wherein FIG. 2A to FIG. 2E correspond to Comparative Example 1, and FIG. 2F to FIG. 2J correspond to Examples 4. It can be seen from FIG. 2F to FIG. 2J that when the hydrophobic and self-cleaning resin dental material according to Example 4 was lifted up by hands and the pressure was applied to the water droplet, the water droplet was deformed; and when the resin dental material was moved downward, the water droplet did not adhere to the material surface. The air layer on the surface of the hydrophobic resin dental material could reduce the contact area between the sample surface and water, thereby reducing an adhesion with water and playing the role of hydrophobic and self-cleaning effects. It can be seen from FIG. 2A to FIG. 2E that during lifting the hydrophilic resin dental material upwards by hands, the water droplet adhered to the surface and spread out immediately after contacting the material, indicating a strong adhesion between the hydrophilic resin dental material and water.

### (3) Biocompatibility

A test method: the hydrophobic and self-cleaning resin dental material according to Example 4, the hydrophilic resin dental material according to Comparative Example 1, and the cured commercial resin dental material according to Comparative Example 2 were separately placed in 85% DMEM+15% fetal bovine serum, and then extracted to obtain the corresponding extracts to be tested for each material. The 85% DMEM+15% fetal bovine serum was used as a blank control.

Mouse osteogenic precursor cells (MC3T3-E1) were co-cultured with each of the extracts to be tested for 1 d, 3 d, and 5 d using an extraction solution method, and then stained with rhodamine 123, and cell growth and cell proliferation were observed with a fluorescence microscope.

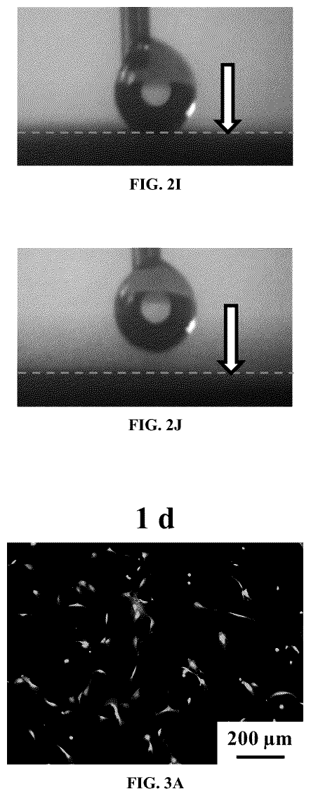

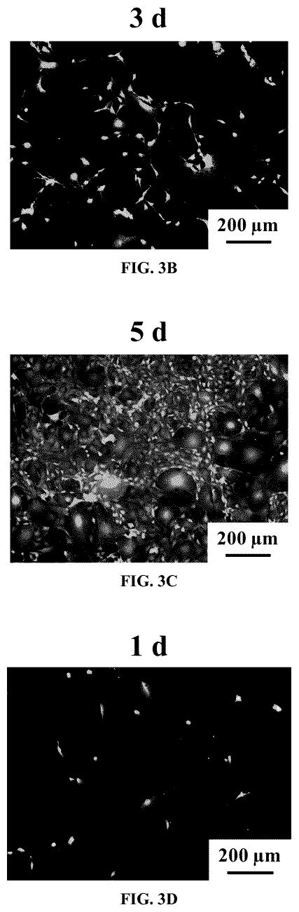

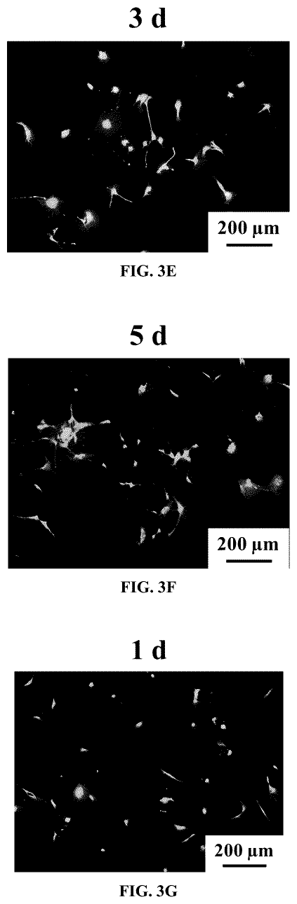

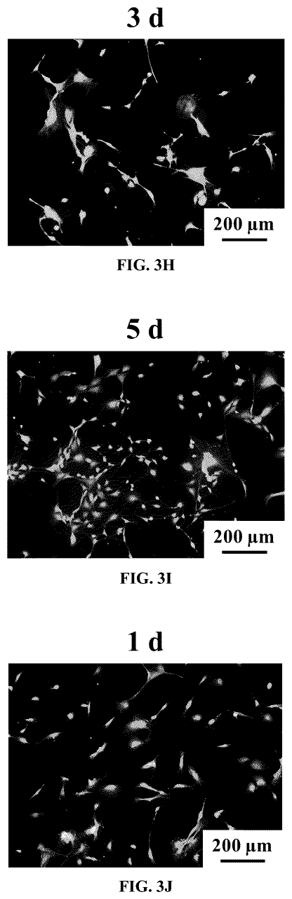

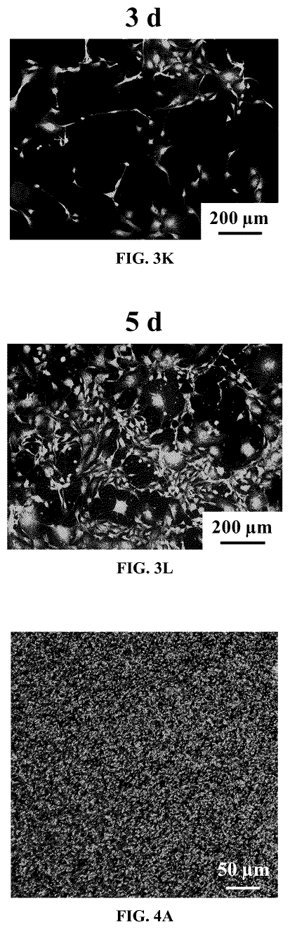

FIG. 3A to FIG. 3L show fluorescent micrographs of the cell compatibility of extract solutions of the resin dental materials according to Example 4 and Comparative Examples 1 to 2 and a blank control group, wherein FIG. 3A to FIG. 3C correspond to the blank control group, FIG. 3D to FIG. 3F correspond to Comparative Example 2, FIG. 3G to FIG. 3I correspond to Comparative Example 1, and FIG. 3J to FIG. 3L correspond to Example 4. From FIG. 3J to FIG. 3L, it can be seen that in the experimental group co-cultured with the extract of the hydrophobic and self-cleaning resin dental material according to Example 4, the cells have the highest proliferation density and the best shape, which are better than the blank control group and the hydrophilic resin dental material group (Comparative Example 1), and even far better than the commercial resin dental material group (Comparative Example 2). It shows that compared with the two existing resin dental materials and the blank control group, the hydrophobic and self-cleaning resin dental material provided by the present disclosure has desirable biocompatibility.

### (4) Bacterial Adhesion on the Surface

A test method: the hydrophobic and self-cleaning resin dental material according to Example 4, the hydrophilic resin dental material according to Comparative Example 1, and the cured commercial resin dental material according to Comparative Example 2 were separately co-cultured with Streptococcus mutans for 6 h, subjected to sampling, rinsing, and staining of live/dead bacteria, and the adhesion of bacteria on the sample surface was observed with a laser scanning confocal microscope. A group without the resin dental materials were used as blank control group.

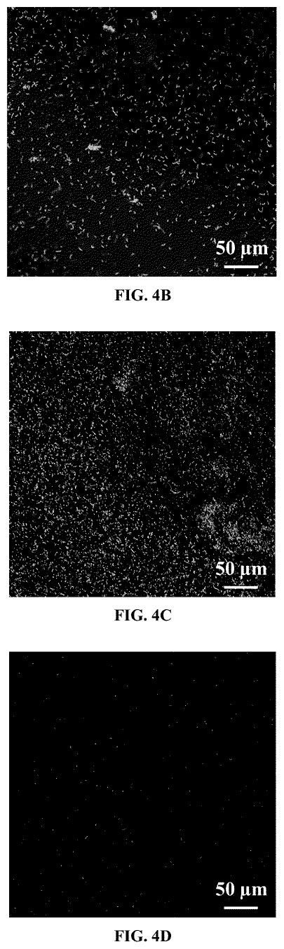

FIG. 4A to FIG. 4D show fluorescence staining images of bacterial adhesion on the surfaces of the resin dental materials according to Example 4 and Comparative Examples 1 to 2 and the blank control group by a laser scanning confocal microscope, wherein FIG. 4A corresponds to the blank control group, FIG. 4B corresponds to the Comparative Example 2, FIG. 4C corresponds to the Comparative Example 1, and FIG. 4D corresponds to the Example 4. It can be seen from FIG. 4D that only a very small amount of bacteria adhered to the surface of the hydrophobic and self-cleaning resin dental material according to Example 4, proving that the material has a desirable performance in inhibiting bacterial adhesion, that is, the “anti-biofouling” performance; while surfaces of the blank control group, the commercial resin dental material group (Comparative Example 2), and the hydrophilic resin dental material group (Comparative Example 1) all adhered to a large number of Streptococcus mutans to varying degrees. It shows that compared with the two existing resin dental materials and the blank control group, the hydrophobic and self-cleaning resin dental material provided by the present disclosure could effectively inhibit the adhesion of bacteria, showing an excellent self-cleaning performance of “anti-biofouling”.

In summary, the hydrophobic and self-cleaning resin dental material has a lotus leaf-like micro-nano structure, excellent hydrophobicity, desirable biocompatibility, and self-cleaning performance of “anti-biofouling” that inhibits bacterial adhesion. The material can be applied to the surface of teeth, dentures, self-cleaning coating of tooth accessories for oral treatment, restorations, implant surfaces, and some special oral environments, such as prevention and treatment of dental caries in patients with senile root caries and xerostomia.

The above descriptions are merely preferred embodiments of the present disclosure. It should be noted that a person of ordinary skill in the art may further make several improvements and modifications without departing from the principle of the present disclosure, but such improvements and modifications should be deemed as falling within the protection scope of the present disclosure.

1-13. (canceled)

14. A hydrophobic and self-cleaning resin slurry, comprising the following raw materials in parts by weight: 3 parts to 10 parts of a dimethacrylate monomer, 5 parts to 30 parts of triethylene glycol dimethacrylate (TEGDMA), 5 parts to 15 parts of perfluoroalkyl acrylate (FMA), 0 part to 15 parts of a diluent, 1 part to 15 parts of nano-silica, 1 part to 30 parts of γ-methacryloxypropyltrimethoxysilane (KH-570), 0.05 parts to 0.5 parts of camphorquinone (CQ), and 0.1 parts to 1 part of a photo-curing accelerator.

15. The hydrophobic and self-cleaning resin slurry of claim 14, wherein the dimethacrylate monomer comprises one or more selected from the group consisting of bisphenol A-glycerolate dimethacrylate (Bis-GMA) and urethane dimethacrylate (UDMA).

16. The hydrophobic and self-cleaning resin slurry of claim 14, wherein the photo-curing accelerator comprises one or more selected from the group consisting of ethyl 4-dimethylaminobenzoate (EDMAB) and dimethylaminoethyl methacrylate (DMAEMA).

17. The hydrophobic and self-cleaning resin slurry of claim 14, wherein the nano-silica has a particle size of 15 nm to 20 nm.

18. The hydrophobic and self-cleaning resin slurry of claim 14, wherein the diluent is an alcohol solvent.

19. A method for preparing the hydrophobic and self-cleaning resin slurry of claim 14, comprising the following steps:
mixing the dimethacrylate monomer, the TEGDMA, the FMA, the diluent, the nano-silica, the KH-570, the CQ, and the photo-curing accelerator to obtain the hydrophobic and self-cleaning resin slurry.

20. The method of claim 19, wherein the mixing is conducted for 1.5 h to 6 h.

21. The method of claim 19, wherein the mixing comprises:
conducting a first mixing on the dimethacrylate monomer, the TEGDMA, the FMA, and the diluent to obtain a first mixture;
conducting a second mixing on the first mixture, the nano-silica, and the KH-570 to obtain a second mixture; and
conducting a third mixing on the second mixture, the CQ, and the photo-curing accelerator.

22. The method of claim 21, wherein the first mixing is conducted for 0.5 h to 2 h; the second mixing is conducted for 0.5 h to 2 h; and the third mixing is conducted for 0.5 h to 2 h.

23. A hydrophobic and self-cleaning resin dental material, obtained by conducting photo-curing on the hydrophobic and self-cleaning resin slurry of claim 14.

24. The hydrophobic and self-cleaning resin dental material of claim 23, wherein the photo-curing is conducted at an optical wavelength of 420 nm to 480 nm.

25. The hydrophobic and self-cleaning resin dental material of claim 23, wherein the photo-curing is conducted for 20 s to 80 s.

26. The method of claim 20, wherein the mixing comprises:
conducting a first mixing on the dimethacrylate monomer, the TEGDMA, the FMA, and the diluent to obtain a first mixture;
conducting a second mixing on the first mixture, the nano-silica, and the KH-570 to obtain a second mixture; and
conducting a third mixing on the second mixture, the CQ, and the photo-curing accelerator.

27. The hydrophobic and self-cleaning resin dental material of claim 23, wherein the material is obtained by conducting photo-curing on the hydrophobic and self-cleaning resin slurry of claim 15.

28. The hydrophobic and self-cleaning resin dental material of claim 23, wherein the material is obtained by conducting photo-curing on the hydrophobic and self-cleaning resin slurry of claim 16.

29. A hydrophobic and self-cleaning resin dental material, obtained by conducting photo-curing on the hydrophobic and self-cleaning resin slurry prepared by the method of claim 19.

30. The hydrophobic and self-cleaning resin dental material of claim 24, wherein the photo-curing is conducted for 20 s to 80 s.

31. The method of claim 19, wherein the dimethacrylate monomer comprises one or more selected from the group consisting of bisphenol A-glycerolate dimethacrylate (Bis-GMA) and urethane dimethacrylate (UDMA).

32. The method of claim 19, wherein the photo-curing accelerator comprises one or more selected from the group consisting of ethyl 4-dimethylaminobenzoate (EDMAB) and dimethylaminoethyl methacrylate (DMAEMA).
